(19) United States
(12) Patent Application Publication
(10) Pub. No.: US 2025/0266151 A1
(43) Pub. Date: Aug. 21, 2025

(54) AUTOMATIC STANDARDIZATION DEVICE, METHOD, AND PROGRAM FOR CLINICAL TRIAL IMAGE BASED ON ARTIFICIAL INTELLIGENCE

(71) Applicant: TRIAL INFORMATICS INC., Seoul (KR)

(72) Inventors: Kyung Won KIM, Seoul (KR); Jung Hyun LEE, Hanam-si (KR); Yousun KO, Seongnam-si (KR); Yong Bin SHIN, Seoul (KR)

(73) Assignee: TRIAL INFORMATICS INC., Seoul (KR)

(21) Appl. No.: 19/203,150

(22) Filed: May 8, 2025

Related U.S. Application Data
(63) Continuation of application No. PCT/KR2023/017835, filed on Nov. 8, 2023.

(30) Foreign Application Priority Data
Nov. 8, 2022 (KR) 10-2022-0148095
Nov. 6, 2023 (KR) 10-2023-0152146

(51) Int. Cl. G16H 30/20; G06T 7/00; G06V 10/764; G06V 10/82; G16H 10/20; G16H 30/40

(52) U.S. Cl. CPC G16H 30/20; G06T 7/0012; G06V 10/764; G06V 10/82; G16H 10/20; G16H 30/40; G06T 2207/20081; G06T 2207/20084; G06V 2201/03

(57) ABSTRACT

Disclosed is an automatic standardization device for a clinical trial image, and the device extracts information for at least one attribute from medical standard data in the clinical trial image, and obtains a first output result based on the extracted information using the extracted information and a pre-learned first artificial intelligence model, obtains a second output result by analyzing the clinical trial image based on an image using a pre-learned second artificial intelligence model, and standardizes the clinical trial image based on the first output result and the second output result.

### CROSS-REFERENCE TO RELATED APPLICATIONS

The present application is a continuation of International Patent Application No. PCT/KR2023/017835, filed on Nov. 8, 2023, which is based upon and claims the benefit of priority to Korean Patent Application No. 10-2023-0152146, filed on Nov. 6, 2023, and to Korean Patent Application No. 10-2022-0148095, filed on Nov. 8, 2022. The disclosures of the above-listed applications are hereby incorporated by reference herein in their entirety.

### BACKGROUND

### 1. Technical Field

The present disclosure relates to a device capable of automatically standardizing a clinical trial image, and more specifically, to a device capable of automatically classifying and standardizing a clinical trial image based on artificial intelligence.

### 2. Description of Related Art

As the use of medical images with advanced technology and complex protocols increases in multi-institutional clinical trials, the importance of standardization of imaging devices and acquisition protocols is increasing.

However, there is a problem that a lot of time and manpower are consumed as the process of manually verifying whether clinical trial image data is standardized, organizing errors and corrections, and then correcting them again is repeated.

Accordingly, a technology capable of automatically classify and standardize clinical trial images is needed, but at present, a technology capable of automatically classify and standardize clinical trial images accurately while minimizing manpower consumption has not been disclosed.

### SUMMARY

The embodiment disclosed in this disclosure is intended to automatically standardize a clinical trial image from various institutions that are not standardized by analyzing the clinical trial image based on an artificial intelligence model.

Technical problems of the inventive concept are not limited to the technical problems mentioned above, and other technical problems not mentioned will be clearly understood by those skilled in the art from the following description.

In an aspect of the present disclosure, an automatic standardization device for a clinical trial image based on artificial intelligence may include a reception module configured to receive a clinical trial image; a memory configured to store at least one instruction; and a processor, wherein the processor is configured to execute the at least one instruction to: extract information for at least one attribute from medical standard data in the clinical trial image, and obtain a first output result based on the extracted information using a pre-learned first artificial intelligence model, obtain a second output result by analyzing the clinical trial image based on an image using a pre-learned second artificial intelligence model, and standardize the clinical trial image based on the first output result and the second output result.

Furthermore, the medical standard data may be a data related to digital imaging and communications in medicine (DICOM) standard, and the processor may be configured to extract the information for at least one attribute from a header of the DICOM.

Furthermore, the first artificial intelligence model may be learned through rule-based labeling using the extracted information.

Furthermore, the first artificial intelligence model may be learned through rule-based labeling using information extracted for a first attribute among the at least one attribute.

Furthermore, the processor may be configured to perform a data standardization for information extracted for a second attribute other than the first attribute among the at least one attribute, and input the data to the first artificial intelligence model.

Furthermore, the first attribute may include at least one attribute that has a probability to include a keyword related to at least one sequence type of the clinical trial image.

Furthermore, the rule may include a first rule table and a second rule table, and the first artificial intelligence model may be configured to, based on the extracted information including one sequence type in the extracted information, perform labeling based on the first rule table, and, based on the extracted information includes multiple sequence types in the extracted information, perform labeling based on the second rule table.

Furthermore, the first output result may include a classification result for the sequence type for the clinical trial image, and the first artificial intelligence model may be configured to generate the first output result based on a random forest algorithm.

Furthermore, the processor may be configured to obtain the second output result by performing a majority vote on a classification result for the sequence type based on the clinical trial image using the second artificial intelligence model.

Furthermore, the processor may be configured to, based on the first output result and the second output result being not identical, check whether an amount of the medical standard data in the clinical trial image satisfies a preset condition, and based on the amount of the medical standard data not satisfying the preset condition, standardize the clinical trial image based on the second output result.

Furthermore, the processor may be configured to, based on the amount of the medical standard data satisfying the preset condition, and based on the sequence type included in the first output result including Perfusion, standardize the clinical trial image based on the second output result.

In another aspect of the present disclosure, an automatic standardization method for a clinical trial image based on artificial intelligence performed by a device may include extracting information for at least one attribute from medical standard data in the clinical trial image; obtaining a first output result based on the extracted information using a pre-learned first artificial intelligence model; obtaining a second output result by analyzing the clinical trial image based on an image using a pre-learned second artificial intelligence model; and standardizing the clinical trial image based on the first output result and the second output result.

In addition, a computer program stored in a computer-readable recording medium for implementing the present disclosure may be further provided.

In addition, a computer-readable recording medium recording a computer program for implementing the present disclosure may be further provided.

### BRIEF DESCRIPTION OF THE FIGURES

FIG. 1 is a schematic diagram of an automatic standardization system for a clinical trial image based on artificial intelligence according to an embodiment of the present disclosure.

FIG. 2 is a block diagram of the automatic standardization device for a clinical trial image based on artificial intelligence according to an embodiment of the present disclosure.

FIG. 3 is a diagram illustrating data stored in the memory of the automatic standardization device for a clinical trial image based on artificial intelligence according to an embodiment of the present disclosure.

FIG. 4 is a diagram illustrating a standardization process for a clinical trial image.

FIG. 5 is a diagram illustrating an example of performing automatic standardization using an artificial intelligence model for a standardization process similar to FIG. 4.

FIG. 6 is a flowchart of an automatic standardization method for a clinical trial image based on artificial intelligence according to an embodiment of the present disclosure.

FIG. 7 is a diagram illustrating classification of Sequence Type from DICOM Header of a clinical trial image.

FIG. 8 is a diagram illustrating extraction of DICOM attribute from DICOM Header of a clinical trial image.

FIG. 9 is a diagram illustrating an example of a specific keyword included in an attribute extracted from a DICOM Header.

FIG. 10 is a diagram illustrating an example of a rule table for automatic labeling.

FIG. 11 is a diagram illustrating a first artificial intelligence model training.

FIG. 12 is a diagram illustrating obtaining the second output result using the image-based second artificial intelligence model.

FIG. 13 is a diagram illustrating the accuracy of the output result obtained using the first artificial intelligence model.

FIG. 14 is a diagram illustrating the accuracy of the output result obtained using the second artificial intelligence model.

FIG. 15 is a diagram illustrating the process of ensembling the output result obtained using the first artificial intelligence model and the second artificial intelligence model.

### DETAILED DESCRIPTION

In the drawings, the same reference numeral refers to the same element. This disclosure does not describe all elements of embodiments, and general contents in the technical field to which the present disclosure belongs or repeated contents of the embodiments will be omitted. The terms, such as “unit, module, member, and block” may be embodied as hardware or software, and a plurality of “units, modules, members, and blocks” may be implemented as one element, or a unit, a module, a member, or a block may include a plurality of elements.

Throughout this specification, when a part is referred to as being “connected” to another part, this includes “direct connection” and “indirect connection”, and the indirect connection may include connection via a wireless communication network.

Furthermore, when a certain part “includes” a certain element, other elements are not excluded unless explicitly described otherwise, and other elements may in fact be included.

In the entire specification of the present disclosure, when any member is located “on” another member, this includes a case in which still another member is present between both members as well as a case in which one member is in contact with another member.

The terms “first,” “second,” and the like are just to distinguish an element from any other element, and elements are not limited by the terms.

The singular form of the elements may be understood into the plural form unless otherwise specifically stated in the context.

Identification codes in each operation are used not for describing the order of the operations but for convenience of description, and the operations may be implemented differently from the order described unless there is a specific order explicitly described in the context.

The operating principle and embodiments of the present disclosure are described below with reference to the attached drawings.

In this specification, the term ‘automatic standardization device for a clinical trial image according to the present disclosure’ includes all of various devices that can perform computational processing and provide results to the user. For example, the device may include all of a computer, a server device, and a portable terminal, or may be in the form of one of them.

Here, the computer may include, for example, a notebook, a desktop, a laptop, a tablet PC, a slate PC, and the like mounted with a web browser.

The server device is a server that communicates with an external device to process information, and may include an application server, a computing server, a database server, a file server, a mail server, a proxy server, and a web server.

A portable terminal is a wireless communication device that ensures portability and mobility, and may include all kinds of handheld-based wireless communication devices such as PCS (Personal Communication System), GSM (Global System for Mobile communications), PDC (Personal Digital Cellular), PHS (Personal Handyphone System), PDA (Personal Digital Assistant), IMT (International Mobile Telecommunication)-2000, CDMA (Code Division Multiple Access)-2000, W-CDMA (W-Code Division Multiple Access), WiBro (Wireless Broadband Internet) terminal, a smart phone, and the like, and a wearable device such as at least one of a watch, a ring, bracelets, anklets, a necklace, glasses, contact lenses, or a head-mounted device (HMD).

The function related to artificial intelligence according to the present disclosure operates through a processor and a memory. The processor may be composed of one or more processors. At this time, the one or more processors may be a general-purpose processor such as a CPU, an AP, a DSP (Digital Signal Processor), a graphics-only processor such as a GPU, a VPU (Vision Processing Unit), or an artificial intelligence-only processor such as an NPU. The one or more processors control input data to be processed according to a predefined operation rule or artificial intelligence model stored in the memory. Alternatively, in the case that the one or more processors are artificial intelligence-only processors, the artificial intelligence-only processor may be designed as a hardware structure specialized for processing a specific artificial intelligence model.

The predefined operation rule or artificial intelligence model may be created through learning. Here, being created through learning means that a basic artificial intelligence model is learned by using a plurality of learning data by a learning algorithm, thereby creating a predefined operation rule or artificial intelligence model set to perform a desired feature (or, purpose). Such learning may be performed on the device itself in which the artificial intelligence according to the present disclosure is performed, or may be performed through a separate server and/or system. Examples of learning algorithms include supervised learning, unsupervised learning, semi-supervised learning, or reinforcement learning, but are not limited to the examples described above.

The artificial intelligence model may include a plurality of neural network layers. Each of the plurality of neural network layers has a plurality of weights, and performs neural network operations through operations between the operation results of the previous layer and the plurality of weights. The plurality of weights of the plurality of neural network layers may be optimized by the learning results of the artificial intelligence model. For example, the plurality of weights may be updated so that the loss value or cost value acquired by the artificial intelligence model is reduced or minimized during the learning process. The artificial neural network may include a deep neural network (DNN), for example, a convolutional neural network (CNN), a deep neural network (DNN), a recurrent neural network (RNN), a restricted Boltzmann machine (RBM), a deep belief network (DBN), a bidirectional recurrent deep neural network (BRDNN), or a deep Q-network, but is not limited to the examples described above.

According to an exemplary embodiment of the present disclosure, the processor may implement artificial intelligence. Artificial intelligence refers to a machine learning method based on an artificial neural network that imitates human neurons (biological neurons) to enable a machine to learn. The artificial intelligence methodology may be divided into supervised learning in which input data and output data are provided together as training data according to a learning method so that the answer (output data) to a problem (input data) is determined, unsupervised learning in which only input data is provided without output data so that the answer (output data) to a problem (input data) is not determined, and reinforcement learning in which a reward is given from an external environment whenever an action is taken in a current state (State), and learning is performed in a direction to maximize this reward. In addition, the methodology of artificial intelligence can be classified according to the architecture, which is the structure of the learning model. The architecture of widely used deep learning technology can be classified into convolutional neural network (CNN), recurrent neural network (RNN), transformer, and generative adversarial network (GAN).

The present device may include an artificial intelligence model. The artificial intelligence model may be one artificial intelligence model or may be implemented as multiple artificial intelligence models. The artificial intelligence model may be composed of a neural network (or artificial neural network) and may include a statistical learning algorithm that mimics the neurons of biology in machine learning and cognitive science. A neural network may mean an overall model that has problem-solving capabilities by changing the strength of the synapse connection through learning by forming a network with artificial neurons (nodes) that combine synapses. The neurons of the neural network may include a combination of weights or biases. The neural network may include one or more layers composed of one or more neurons or nodes. For example, the device may include an input layer, a hidden layer, and an output layer. The neural network constituting the device can infer a desired result (output) from an arbitrary input (input) by changing the weights of neurons through learning.

The processor may generate a neural network, train (or learn) a neural network, perform a calculation based on received input data, generate an information signal based on the result of the calculation, or retrain the neural network. The models of the neural network may include various types of models such as CNN (Convolution Neural Network) such as GoogleNet, AlexNet, VGG Network, R-CNN (Region with Convolution Neural Network), RPN (Region Proposal Network), RNN (Recurrent Neural Network), S-DNN (Stacking-based deep Neural Network), S-SDNN (State-Space Dynamic Neural Network), Deconvolution Network, DBN (Deep Belief Network), RBM (Restrcted Boltzman Machine), Fully Convolutional Network, LSTM (Long Short-Term Memory) Network, Classification Network, and the like, but are not limited thereto. The processor may include one or more processors for performing calculations according to the models of the neural network. For example, a neural network may include a deep neural network.

The neural network may include CNN (Convolutional Neural Network), RNN (Recurrent Neural Network), percept, multilayer perceptron, FF (Feed Forward), RBF (Radial Basis Network), DFF (Deep Feed Forward), LSTM (Long Short Term Memory), Gated Recurrent Unit (GRU), Auto Encoder (AE), Variational Auto Encoder (VAE), Denoising Auto Encoder (DAE), Sparse Auto Encoder (SAE), Markov Chain (MC), Hopfield Network (HN), Boltzmann Machine (BM), Restricted Boltzmann Machine (RBM), Depp Belief Network (DBN), Deep Convolutional Network (DCN), Deconvolutional Network (DN), Deep Convolutional Inverse Graphics Network (DCIGN), Generative Adversarial Network (GAN), Liquid State Machine (LSM), Extreme Learning Machine (ELM), Echo State Network (ESN), Deep Residual Network (DRN), Differentiable Neural Computer (DNC), Neural Turning Machine (NTM), Capsule Network (CN), Kohonen Network (KN), and Attention Network (AN), but not limited thereto, and it will be understood by those skilled in the art that any neural network may be included.

According to an exemplary embodiment of the present disclosure, the processor may use various artificial intelligence structures and algorithms such as CNN (Convolution Neural Network), R-CNN (Region with Convolution Neural Network), RPN (Region Proposal Network), RNN (Recurrent Neural Network), S-DNN (Stacking-based deep Neural Network), S-SDNN (State-Space Dynamic Neural Network), Deconvolution Network, DBN (Deep Belief Network), RBM (Restricted Boltzmann Machine), Fully Convolutional Network, LSTM (Long Short-Term Memory) Network, Classification Network, Generative Modeling, eXplainable AI, Continual AI, Representation Learning, and A I for Material Design such as GoogleNet, AlexNet, VGG Network, BERT, SP-BERT, MRC/QA, Text Analysis, Dialog System, GPT-3, and GPT-4 for natural language processing, Visual Analytics, Visual Understanding, Video Synthesis for vision processing, Anomaly Detection, Prediction, Time-Series Forecasting, Optimization, and Recommendation for algorithms ResNet for data intelligence, but not limited thereto. Hereinafter, the embodiment of the present disclosure will be described in detail.

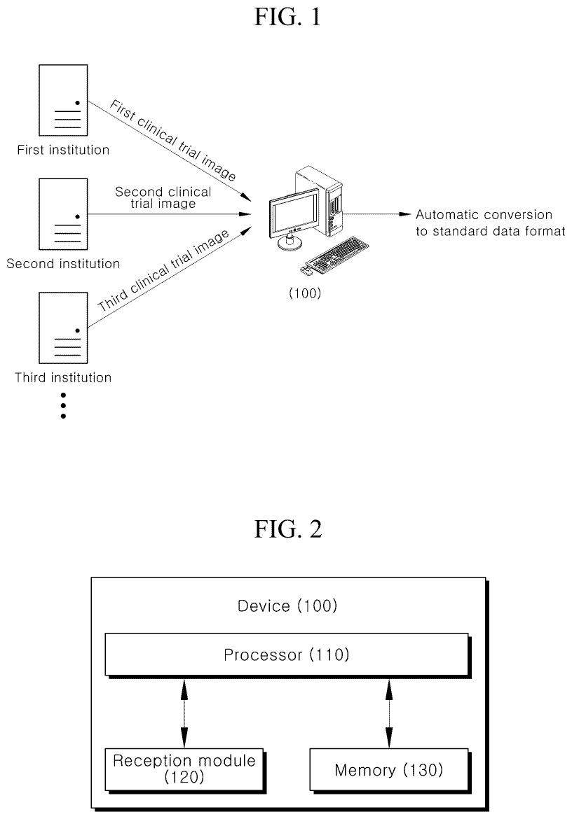

FIG. 1 is a schematic diagram of an automatic standardization system for a clinical trial image based on artificial intelligence according to an embodiment of the present disclosure.

Referring to FIG. 1, an automatic standardization system for a clinical trial image based on artificial intelligence according to an embodiment of the present disclosure is illustrated.

An automatic standardization device 100 for a clinical trial image based on artificial intelligence receives clinical trial images from multiple institutions, and the received clinical trial images are not standardized.

Conventionally, a lot of time and manpower are consumed as the process of manually verifying whether the clinical trial image data received in this way is standardized, the organizing error and correction, and then repeating the process of correcting them again is repeated.

However, through the automatic standardization device 100 for a clinical trial image based on artificial intelligence according to an embodiment of the present disclosure, which will be described below, standardization of clinical trial images is automatically performed.

The automatic standardization device 100 for a clinical trial image based on artificial intelligence according to an embodiment of the present disclosure is configured to include a server device and may operate as an automatic standardization server for a clinical trial image based on artificial intelligence.

Hereinafter, with reference to other drawings, the automatic standardization device 100, method, and program for a clinical trial image based on artificial intelligence according to an embodiment of the present disclosure will be described in detail.

FIG. 2 is a block diagram of the automatic standardization device 100 for a clinical trial image based on artificial intelligence according to an embodiment of the present disclosure.

Referring to FIG. 2, the automatic standardization device 100 for a clinical trial image based on artificial intelligence according to an embodiment of the present disclosure includes a processor 110, a reception module 120, and a memory 130.

However, in some embodiments, the device 100 may include fewer or more components than those illustrated in FIG. 2.

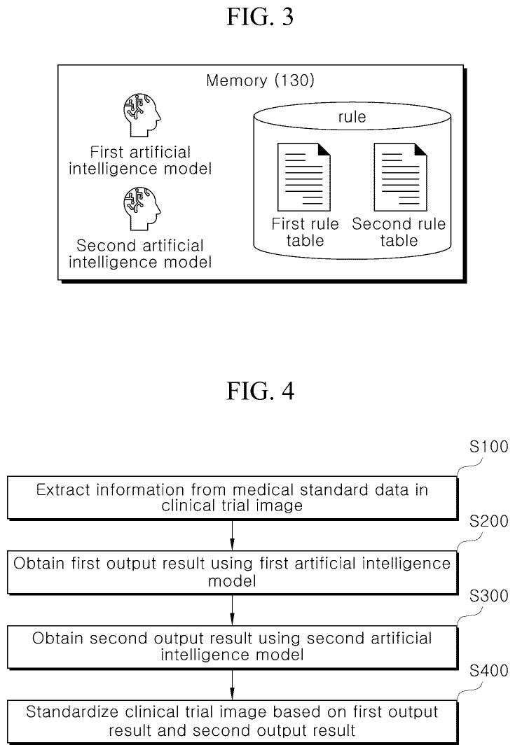

FIG. 3 is a diagram illustrating data stored in the memory 130 of the automatic standardization device 100 for a clinical trial image based on artificial intelligence according to an embodiment of the present disclosure.

The processor 110 may be implemented as a storage module storing data for an algorithm for controlling the operation of components within the device 100 or a program that reproduces the algorithm, and at least one processor that performs the above-described operation using the data stored in the storage module. At this time, the storage module and the processor 110 may be implemented as separate chips. Alternatively, the storage module and the processor 110 may be implemented as a single chip.

In addition, the processor 110 may control one or more of the components discussed above in combination to implement various embodiments of the present disclosure described in the diagrams below on the device.

In addition to the operation related to the application program, the processor 110 may typically control the overall operation of the device. The processor 110 may process signals, data, information, and the like input or output through the components discussed above, or may operate an application program stored in the storage module, thereby providing or processing appropriate information or functions to the user.

In addition, the processor 110 may control at least some of the components of the device to operate the application program stored in the storage module. In addition, the processor 110 may operate at least two or more of the components included in the device in combination to drive the application program.

The reception module 120 may include a communication module or may be the communication module itself.

The communication module may include one or more modules that connect the automatic standardization device 100 for a clinical trial image to one or more networks.

The communication module may include one or more components that enable communication with an external device, and may include, for example, at least one of a broadcast reception module, a wired communication module, a wireless communication module, a short-range communication module, or a location information module.

The wired communication module may include various wired communication modules such as a Local Area Network (LAN) module, a Wide Area Network (WAN) module, or a Value Added Network (VAN) module, as well as various cable communication modules such as a Universal Serial Bus (USB), a High Definition Multimedia Interface (HDMI), a Digital Visual Interface (DVI), RS-232 (recommended standard232), power line communication, or plain old telephone service (POTS).

The wireless communication module may include a wireless communication module that supports various wireless communication methods such as a WiFi module, a WiBro (Wireless broadband) module, GSM (Global System for Mobile Communication), CDMA (Code Division Multiple Access), WCDMA (Wideband Code Division Multiple Access), UMTS (Universal Mobile Telecommunications System), TDMA (Time Division Multiple Access), LTE (Long Term Evolution), 4G, 5G, and 6G.

The wireless communication module may include a wireless communication interface that includes an antenna and a transmitter that transmits a communication signal. In addition, the wireless communication module may further include a signal conversion module that modulates a digital control signal output from the processor 110 through the wireless communication interface into an analog wireless signal under the control of the processor 110.

The short-range communication module is for short-range communication, and may support short-range communication by using at least one of Bluetooth, RFID (Radio Frequency Identification), Infrared Data Association (IrDA), UWB (Ultra-Wideband), ZigBee, NFC (Near Field Communication), Wi-Fi (Wireless-Fidelity), Wi-Fi Direct, or Wireless USB (Wireless Universal Serial Bus) technology.

The memory 130 may store data supporting various functions of the device 100. The memory may store a plurality of application programs (or applications) running on the device 100, data for the operation of the device 100, and commands. At least some of these application programs may exist for the basic functions of the device 100. Meanwhile, the application programs may be stored in the memory 130, installed on the device 100, and driven to perform operations (or functions) by the processor 110.

The memory 130 may store data supporting various functions of the device and a program for the operation of the processor, input/output data (e.g., music files, still images, moving images, etc.) may be stored, and a plurality of application programs (or applications) run on the device, data for the operation of the device, and commands can be stored. At least some of these application programs may be downloaded from an external server via wireless communication.

The memory 130 may include at least one type of storage medium among a flash memory 130 type, a hard disk type, an SSD (Solid State Disk type), an SDD (Silicon Disk Drive) type, a multimedia card micro type, a card type memory (for example, an SD or XD memory, etc.), a random access memory (RAM), a static random access memory (SRAM), a read-only memory (ROM), an electrically erasable programmable read-only memory (EEPROM), a programmable read-only memory (PROM), a magnetic memory 130, a magnetic disk, and an optical disk. In addition, the memory 130 may be a database that is separate from the device 100 but connected by wire or wirelessly.

In addition, the memory 130 may have multiple processes for the automatic standardization device 100 for a clinical trial image.

In addition, the automatic standardization device 100 for a clinical trial image may further include components such as an input module, an output module, and an interface module.

The input module is for inputting image information (or signal), audio information (or signal), data, or information input from a user, and may include at least one camera, at least one microphone, and at least one of a user input module. Voice data or image data collected by the input module may be analyzed and processed as a user's control command.

The input module is for receiving information from a user, and when information is input through the input module, the processor 110 may control the operation of the present device 100 to correspond to the input information. The input module 150 may include hardware-type physical keys (e.g., buttons located on at least one of the front, rear, and side of the device, dome switches, jog wheels, jog switches, etc.) and software-type touch keys. As an example, the touch key may be formed as a virtual key, a soft key, or a visual key displayed on a touchscreen-type display module through software processing, or as a touch key placed on a part other than the touchscreen. Meanwhile, the virtual key or visual key may be displayed on the touchscreen in various forms, and may be formed as, for example, a graphic, a text, an icon, a video, or a combination thereof.

The output module is for generating output related to visual, auditory, or tactile sensations, and may include at least one of a display module, an audio output module, a haptic module, or an optical output module. The display module may be formed as a touch sensor and a mutual layer structure or formed as an integral part, thereby implementing a touch screen. This touch screen may function as a user input module that provides an input interface between the device 100 and the user, and may provide an output interface between the device 100 and the user.

The display module displays (outputs) information processed in the device 100. For example, the display module may display execution screen information of an application program for example, an application running in the device 100, or UI (User Interface), GUI (Graphical User Interface) information according to this execution screen information.

The interface module serves as a passageway for various types of external devices connected to the device 100. The interface module may include at least one of a wired/wireless headset port, an external charger port, a wired/wireless data port, a memory card port, a port for connecting a device 100 equipped with an identification module SIM, an audio I/O (Input/Output) port, a video I/O (Input/Output) port, and an earphone port. In the device 100, appropriate control related to an external device connected to the interface unit may be performed.

FIG. 4 is a diagram illustrating a standardization process for a clinical trial image.

Referring to FIG. 4, in the embodiment of the present disclosure, DICOM image data may be applied to the clinical trial image, and a standard protocol may be applied to a CDISC standard data format. In addition, an SDTM dataset may be applied as a dataset for clinical data submission.

SDTM is a domain-based model defined as a standard for submitting clinical trial data.

SDTM is classified into several domains according to the property of clinical trial data, and the clinical trial image automatic classification and standardization device 100 may perform mapping using a data library according to the domain.

The domain of SDTM is a Dataset name that classifies data with common property of data about subjects collected during the clinical trial.

DICOM file format: DICOM (Digital Imaging and Communications in Medicine) is a standard file format for storing and transmitting medical images. A DICOM file includes patient information, image information, and photographing equipment information.

A DICOM image is an abbreviation for Digital Imaging and Communications in Medicine and is a standard format for storing and transmitting medical images. A DICOM image may store images obtained through various medical imaging devices 100 such as X-ray, MRI, CT, and PET. Therefore, in the embodiment of the present disclosure, the clinical trial image may mean a DICOM Image.

SDTM (Study Data Tabulation Model) is a model for standardizing, managing, and analyzing clinical trial data. SDTM is a standard developed and managed by CDISC (Clinical Data Interchange Standards Consortium).

SDTM may classify clinical trial data into three areas as follows:

Interventions: Treatment, drug, device, protocol, and the like.

Events: Events occurring in clinical trials, such as adverse effects, adverse reactions, deaths, and the like.

Findings: Results discovered in clinical trials, such as biological markers, clinical markers, and the like.

SDTM may subdivide each area into the following elements:

Domains: Categories that represent the type of data

Variables: Individual items of data

Levels: Possible values of variables

SDTM provides clear definitions and rules for each element. Through this, SDTM standardizes clinical trial data so that it may be easily exchanged and analyzed across multiple systems.

SDTM helps improve the quality of clinical trial data, increase the reusability of data, and enhance the reliability of clinical trial results.

The main functions of SDTM are as follows:

Standardization of data: SDTM standardizes clinical trial data so that it may be easily exchanged and analyzed across multiple systems.

Reusability of data: SDTM may reuse standardized data for multiple studies.

Improvement of data quality: SDTM improves the quality of data through standardization and rules.

Improving the reliability of clinical trial results: SDTM improves the reliability of clinical trial results through data standardization and rules.

SDTM is an essential standard for managing and analyzing clinical trial data. Using SDTM, the quality and efficiency of clinical trial data may be improved.

The main uses of SDTM are as follows:

Collection of clinical trial data: SDTM may define the structure of the database used to collect clinical trial data.

Analysis of clinical trial data: SDTM may define the data format of the analysis tool used to analyze clinical trial data.

Reporting clinical trial data: SDTM may define the format of the report used to report clinical trial data.

SDTM is a standard for the end-to-end management of clinical trial data. Using SDTM, clinical trial data may be collected, analyzed, and reported effectively.

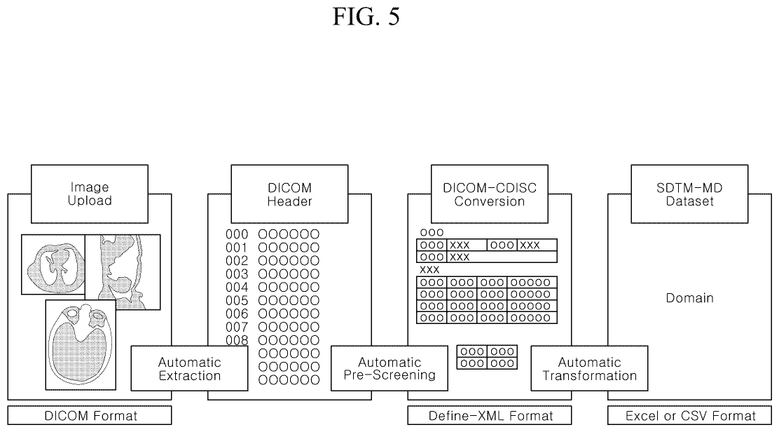

FIG. 5 is a diagram illustrating an example of performing automatic standardization using an artificial intelligence model for a standardization process similar to FIG. 4.

Referring to FIG. 5, the automatic standardization device 100 for a clinical trial image may perform an automatic standardization process for clinical trial images using a first artificial intelligence model based on text analysis and a second artificial intelligence model based on image analysis.

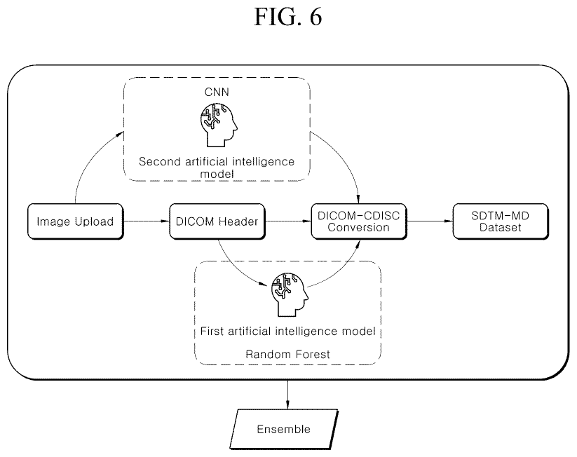

FIG. 6 is a flowchart of an automatic standardization method for a clinical trial image based on artificial intelligence according to an embodiment of the present disclosure.

FIGS. 6 to 15 are various exemplary diagrams for describing the automatic standardization device 100, method and program for a clinical trial image based on artificial intelligence according to an embodiment of the present disclosure.

Hereinafter, the process of the automatic standardization device 100, method and program for a clinical trial image based on artificial intelligence according to an embodiment of the present disclosure will be described in detail with reference to FIGS. 6 to 15.

The processor 110 extracts information from medical standard data in a clinical trial image (step S100).

The processor 110 obtains a first output result using a first artificial intelligence model (step S200).

The processor 110 may further include a step of receiving the clinical trial image through the reception module before step S100.

The processor 110 may extract information for at least one attribute from medical standard data in the clinical trial image.

Then, the processor 110 performs rule-based labeling using the extracted information and obtains a first output result using a pre-learned first artificial intelligence model.

In this case, the medical standard data may be applied with the DICOM (Digital Imaging and Communications in Medicine) standard.

In addition, the clinical trial image may be applied to an MRI image, but is not limited thereto.

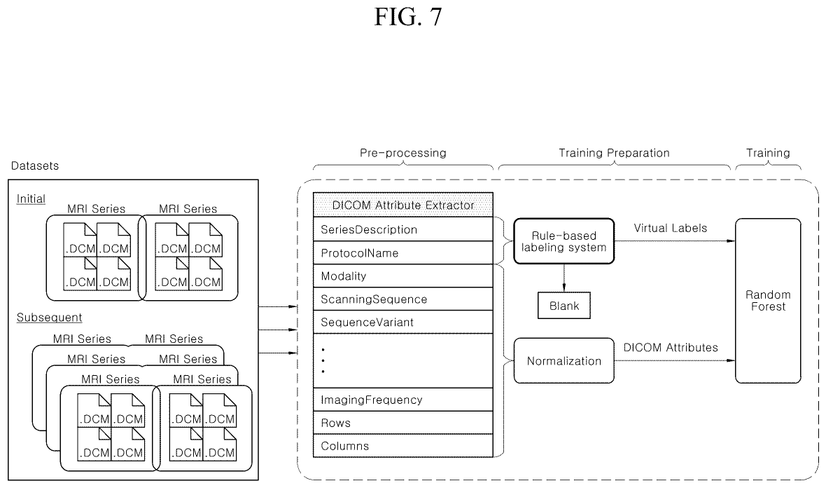

FIG. 7 is a diagram illustrating classification of Sequence Type from DICOM Header of a clinical trial image.

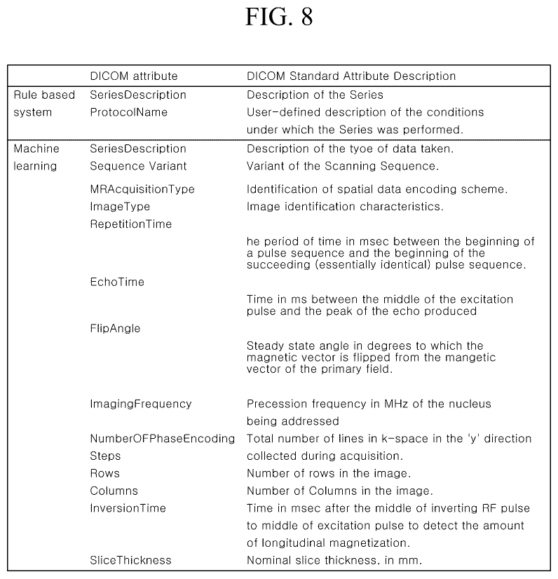

FIG. 8 is a diagram illustrating extraction of DICOM attribute from DICOM Header of a clinical trial image.

Referring to FIG. 7, the processor 110 extracts information for at least one attribute from DICOM Header information of the clinical trial image (MRI image).

At least one attribute is illustrated in FIG. 7 and FIG. 8.

As shown in FIG. 3, the memory 130 stores a preset rule.

The processor 110 performs rule-based labeling using the information extracted from the header of DICOM for the at least one attribute.

The automatic standardization device 100 for a clinical trial image according to the embodiment of the present disclosure uses a Rule-Based System to solve the existing manual labeling problem. The processor 110 trains an artificial intelligence model using the result value of the Rule-Based System as a learning label.

The processor 110 performs rule-based labeling using the information extracted for a first attribute among the at least one attribute.

In this case, the processor 110 performs data normalization for the information extracted for an attribute other than the first attribute (the second attribute) among the at least one attribute and inputs it into the first artificial intelligence model.

Referring to FIG. 7, it is exemplified that rule-based virtual labeling is performed for the SeriesDescription attribute and the ProtocolName attribute among multiple attributes. And, for the remaining attributes, data standardization is performed and a random forest-based process is performed using an artificial intelligence model.

Here, the first attribute includes at least one attribute that has a probability to include a keyword related to at least one Sequence Type for the clinical trial image.

In one embodiment, the first artificial intelligence model is learned through rule-based labeling for the first attribute among the at least one attribute among the information extracted from standard data in the clinical trial image.

In addition, when the processor performs automatic standardization of the clinical trial image, the processor obtains an output result using the pre-learned first artificial intelligence model as described above.

Specifically, the first output result is obtained based on the information extracted from standard data in the clinical trial image using the pre-learned first artificial intelligence model.

In this case, the first artificial intelligence model generates the first output result based on the data of the second attribute among the at least one attribute among the information extracted from standard data in the clinical trial image.

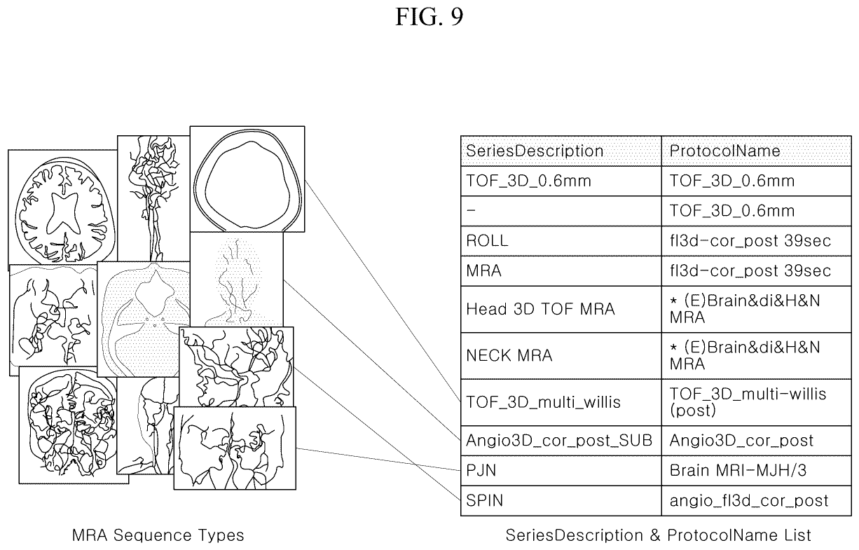

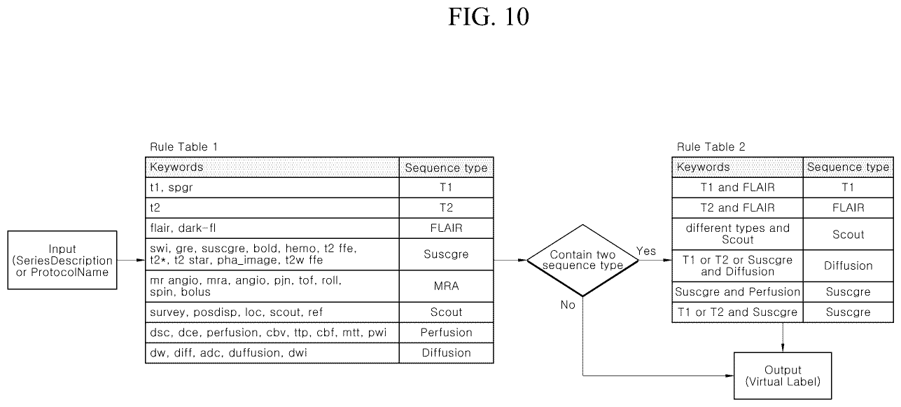

FIG. 9 is a diagram illustrating an example of a specific keyword included in an attribute extracted from a DICOM Header.

Since the SeriesDescription attribute is different depending on the manufacturer or user, there is a problem that it is difficult to infer the SequenceType only with the SeriesDescription attribute.

However, it includes the same or similar keywords to indicate a specific SequenceType.

Referring to FIG. 9, an example of a keyword TOF included in the SeriesDescription attribute and the ProtocolName attribute is shown.

FIG. 10 is a diagram illustrating an example of a rule table for automatic labeling.

Referring to FIG. 10, the automatic standardization device 100 for a clinical trial image may include multiple rule tables.

Rule Table 1 illustrated in FIG. 10 means the first rule table, and Rule Table 2 means the second rule table.

In the case that the information extracted from step S100 includes one Sequence Type, the processor 110 performs labeling based on the first rule table.

Then, in the case that the information extracted from step S100 includes multiple Sequence Types, the processor 110 performs labeling based on the second rule table.

The first rule table is based on the SeriesDescription (SD) value among the DICOM header properties, and in the case that keywords are included in the SD value, the labeling is performed with the relevant sequence type.

However, in the case that two or more different keywords are included and two or more sequence types are inferred, the processor 110 performs labeling based on the second rule table.

The second rule table is a rule that determines the priority when two or more sequence types are included.

For example, referring to FIG. 9, the second rule table performs labeling as T1 when sequence types T1 and FLAIR appear together.

In the case that the SD does not contain a keyword, labeling is not performed.

The Sequence Type will be briefly described.

In medical data, Sequence Type is a term that indicates the type of image obtained through an MRI examination. The MRI examination is a test that shows the internal structure of the human body as an image, and various information may be obtained by using various sequence types.

T1, T2, and Flair are the most basic sequence types used in the MRI examination.

The T1 sequence is a method of obtaining an image by measuring the magnetic rotation time of hydrogen atoms. T1 images are effective in distinguishing tissues such as nerve tissue, bone, and fat.

T2 sequence is a method of obtaining images by measuring the magnetic rotational spread of hydrogen atoms. T2 images are effective in distinguishing tissues such as white matter, fluid, and inflammation in the brain.

The Flair sequence is similar to the T2 image, but is designed to better distinguish the differences between the cortex and ventricles of the brain.

The Suscgre sequence is a method of obtaining images by utilizing the magnetic rotational properties of hydrogen atoms. Suscgre images are effective in distinguishing blood vessels in the brain.

The MRA sequence is methods of observing blood vessels in the brain in more detail using the Suscgre sequence.

The SCOUT sequence is a sequence used to set the examination range by scanning the patient's body before starting an MRI examination.

The Perfusion sequence is a method of obtaining images by measuring blood flow. Perfusion images are used to diagnose diseases such as stroke and cerebral hemorrhage.

The Diffusion sequence is a method of obtaining images by measuring the diffusion speed of hydrogen atoms. Diffusion images are used to diagnose diseases such as stroke and brain tumor.

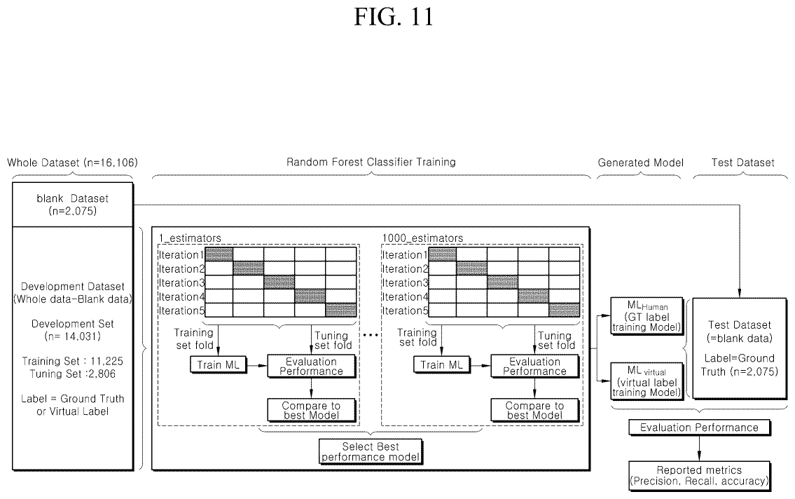

FIG. 11 is a diagram illustrating a first artificial intelligence model training.

Referring to FIG. 11, an ML algorithm training set is configured by excluding data that the Rule Based Labeling System incapable of inferring from the entire dataset.

At this time, the automatic standardization device 100 for a clinical trial image does not use data which is not inferred for learning, so the device may use the data as a test set to evaluate the model.

The automatic standardization device 100 for a clinical trial image may partially utilize human labeling in an initial stage, and when a certain level of accuracy is secured by training the artificial intelligence model with this, it may proceed with automatic labeling entirely.

The first output result includes a classification result for the Sequence Type for the clinical trial image.

The first artificial intelligence model may generate a first output result based on the random forest algorithm.

The processor 110 obtains a second output result using the second artificial intelligence model (step S300).

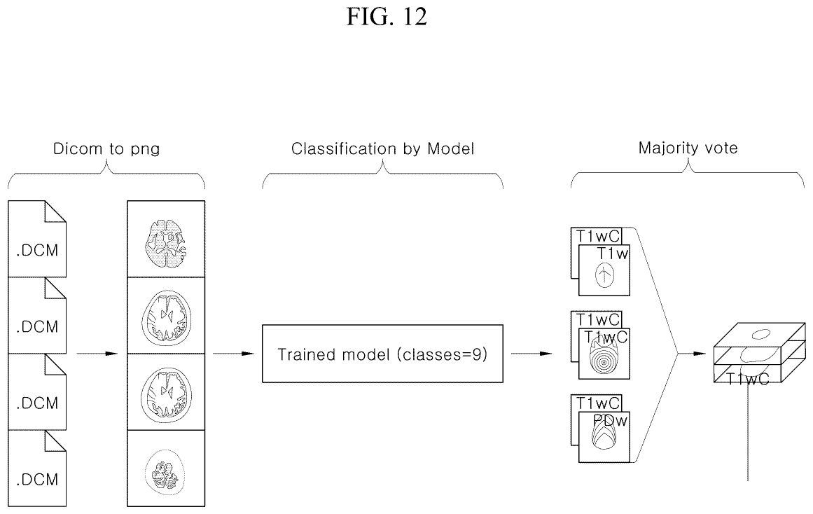

FIG. 12 is a diagram illustrating obtaining the second output result using the image-based second artificial intelligence model.

The second artificial intelligence model may be a model in which an image-based analysis method is learned.

The processor 110 may go through a process of converting the clinical trial image into a preset image format.

To this end, the automatic standardization device 100 for a clinical trial image may further include an image conversion module.

Referring to FIG. 12, the processor 110 converts a DICOM image into a PNG image.

Next, the processor 110 performs data classification using an artificial intelligence model and performs majority voting to obtain the second output result.

That is, the processor 110 obtains the second output result through the following process.

The processor 110 receives a clinical trial image through the communication unit.

The processor 110 converts the clinical trial image format using an image conversion module.

The processor 110 inputs the format-converted image data into a classification model.

The processor 110 obtains an output result including the Sequence Type from the classification model.

The processor 110 performs a majority vote on the output result obtained and obtains the most frequently occurring Type as the second output result.

The processor 110 standardizes the clinical trial image based on the first output result and the second output result (step S400).

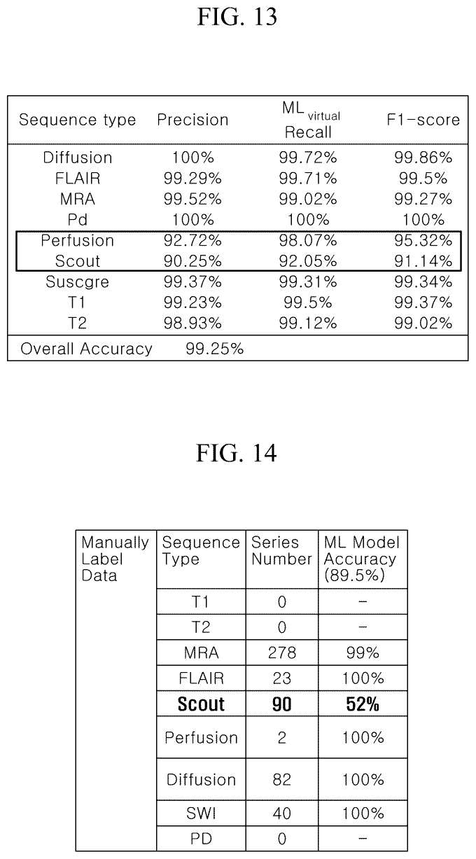

FIG. 13 is a diagram illustrating the accuracy of the output result obtained using the first artificial intelligence model.

FIG. 14 is a diagram illustrating the accuracy of the output result obtained using the second artificial intelligence model.

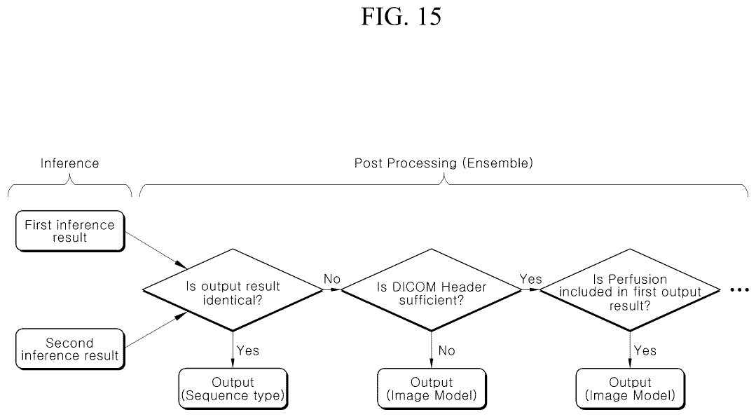

FIG. 15 is a diagram illustrating the process of ensembling the output result obtained using the first artificial intelligence model and the second artificial intelligence model.

The classification accuracy of the text-based first artificial intelligence model may be somewhat lower when there is no attribute value (Feature) of the DICOM header, such as Scout and Perfusion.

The second artificial intelligence model based on the image may have somewhat lower accuracy in the Sequence Type with many changes.

Referring to FIG. 15, in the case that the first output result and the second output result are identical, the processor 110 obtains the first output result or the second output result as the final output value and standardizes the clinical trial image based on this.

In this case, in the case that the first output result and the second output result are not identical, the processor 110 checks whether the clinical trial image has sufficient medical standard data.

Specifically, the processor 110 checks whether an amount of medical standard data (DICOM header) satisfies a preset condition.

Then, in the case that the processor 110 determines that the medical standard data is not sufficient, the processor 110 obtains the second output result of the second artificial intelligence model as the final output value and standardizes the clinical trial image based on this.

The processor 110 obtains the final output value based on the first output result and the second output result, and may select the best value by complementarily using the first output result and the second output result.

For example, in the case that the processor 110 determines that the medical standard data is sufficient, the processor 110 may check whether the first output result includes Perfusion.

Then, in the case that the first output result includes Perfusion, the processor 110 obtains the second output result of the second artificial intelligence model as the final output value, and standardizes the clinical trial image based on this.

The method according to one embodiment of the present disclosure described above may be implemented as a program or application and stored in a medium to be executed in combination with a hardware server.

According to the present disclosure, there is an effect that clinical trial images of various institutions that are not standardized may be automatically standardized by analyzing them based on an artificial intelligence model.

The above-described program may include codes coded in a computer language, such as C, C++, JAVA, or machine language, that may be read by the processor CPU of the computer through the device interface of the computer, so that the computer reads the program and executes the methods implemented as the program. Such codes may include functional codes related to functions that define necessary functions for executing the methods, and may include control codes related to execution procedures necessary for the processor of the computer to execute the functions according to a predetermined procedure. In addition, the code may further include a memory reference-related code regarding which location address of the internal or external memory of the computer should be referenced for additional information or media required for the processor of the computer to execute the functions. In addition, if the processor of the computer needs to communicate with any other computer or server located remotely to execute the functions, the code may further include a communication-related code regarding how to communicate with any other computer or server located remotely using the communication module of the computer, what information or media should be sent and received during the communication, and the like.

The medium in which the storage is performed means a medium that semi-permanently stores data and may be read by a device, rather than a medium that stores data for a short period of time, such as a register, cache, or memory. Specifically, examples of the medium in which the storage is performed include, but are not limited to, ROM, RAM, CD-ROM, magnetic tape, floppy disk, or optical data storage device. That is, the program may be stored in various recording media on various servers that the computer may access or in various recording media on the user's computer. In addition, the media may be distributed to computer systems connected to a network, so that computer-readable codes may be stored in a distributed manner.

The steps of the method or algorithm described in connection with the embodiments of the present disclosure may be implemented directly in hardware, implemented as a software module executed by hardware, or implemented by a combination of these. The software module may reside in random access memory (RAM), read only memory (ROM), erasable programmable ROM (EPROM), electrically erasable programmable ROM (EEPROM), flash memory, a hard disk, a removable disk, a CD-ROM, or any form of computer-readable recording medium well known in the art to which the present disclosure belongs.

Although the embodiments of the present disclosure have been described above with reference to the attached drawings, those skilled in the art will appreciate that the present disclosure may be implemented in other specific forms without changing the technical idea or essential features thereof. Therefore, it should be understood that the embodiments described above are illustrative in all respects and not restrictive.

What is claimed is:

1. An automatic standardization device for a clinical trial image based on artificial intelligence, comprising:
a reception module configured to receive a clinical trial image;
a memory configured to store at least one instruction; and
a processor,
wherein the processor is configured to execute the at least one instruction to:
extract information for at least one attribute from medical standard data in the clinical trial image, and obtain a first output result based on the extracted information using a pre-learned first artificial intelligence model,
obtain a second output result by analyzing the clinical trial image based on an image using a pre-learned second artificial intelligence model, and
standardize the clinical trial image based on the first output result and the second output result.

2. The device according to claim 1,
wherein the medical standard data is a data related to digital imaging and communications in medicine (DICOM) standard, and
wherein the processor is configured to extract the information for at least one attribute from a header of the DICOM.

3. The device according to claim 2,
wherein the first artificial intelligence model is learned through rule-based labeling using the extracted information.

4. The device according to claim 3,
wherein the first artificial intelligence model is learned through rule-based labeling using information extracted for a first attribute among the at least one attribute.

5. The device according to claim 4,
wherein the processor is configured to:
perform a data standardization for information extracted for a second attribute other than the first attribute among the at least one attribute, and input the data to the first artificial intelligence model.

6. The device according to claim 4,
wherein the first attribute includes at least one attribute that has a probability to include a keyword related to at least one sequence type of the clinical trial image.

7. The device according to claim 6,
wherein the rule includes a first rule table and a second rule table, and
wherein the first artificial intelligence model is configured to:
based on the extracted information including one sequence type in the extracted information, perform labeling based on the first rule table, and,
based on the extracted information includes multiple sequence types in the extracted information, perform labeling based on the second rule table.

8. The device according to claim 5,
wherein the first output result includes a classification result for the sequence type for the clinical trial image, and
wherein the first artificial intelligence model is configured to generate the first output result based on a random forest algorithm.

9. The device according to claim 1,
wherein the processor is configured to:
obtain the second output result by performing a majority vote on a classification result for the sequence type based on the clinical trial image using the second artificial intelligence model.

10. The device according to claim 1,
wherein the processor is configured to:
based on the first output result and the second output result being not identical,
check whether an amount of the medical standard data in the clinical trial image satisfies a preset condition, and
based on the amount of the medical standard data not satisfying the preset condition, standardize the clinical trial image based on the second output result.

11. The device according to claim 10,
wherein the processor is configured to:
based on the amount of the medical standard data satisfying the preset condition, and based on the sequence type included in the first output result including Perfusion,
standardize the clinical trial image based on the second output result.

12. An automatic standardization method for a clinical trial image based on artificial intelligence performed by a device, comprising:
extracting information for at least one attribute from medical standard data in the clinical trial image;
obtaining a first output result based on the extracted information using a pre-learned first artificial intelligence model;
obtaining a second output result by analyzing the clinical trial image based on an image using a pre-learned second artificial intelligence model; and
standardizing the clinical trial image based on the first output result and the second output result.

13. The method according to claim 12,
wherein the medical standard data is a data related to digital imaging and communications in medicine (DICOM) standard, and
wherein the processor is configured to extract the information for at least one attribute from a header of the DICOM.

14. The method according to claim 13,
wherein the first artificial intelligence model is learned through rule-based labeling using the extracted information.

15. A computer-readable recording medium storing a program for executing the method of claim 12, coupled with a computer as hardware.
